(19) United States
(12) Patent Application Publication
(10) Pub. No.: US 2026/0363651 A1
(43) Pub. Date: Nov. 19, 2026

(54) TRANSCRANIAL ULTRASOUND DETECTION AND MONITORING OF HYDROCEPHALUS

(71) Applicant: Georgia Tech Research Corporation, Atlanta, GA (US)

(72) Inventors: Konstantinos Arvanitis, Atlanta, GA (US); Pradosh Pritam Dash, Atlanta, GA (US)

(21) Appl. No.: 19/679,559

(22) Filed: May 15, 2026

Related U.S. Application Data
(60) Provisional application No. 63/806,223, filed on May 15, 2025.

(51) Int. Cl. A61B 8/08; A61B 8/00

(52) U.S. Cl. CPC A61B 8/0816; A61B 8/4427; A61B 8/4483; A61B 8/461; A61B 8/5223; A61B 2503/04

(57) ABSTRACT

The present disclosure relates generally to medical diagnostic systems, and more particularly to noninvasive ultrasound-based monitoring of intracranial conditions. A system for monitoring intracranial conditions in a subject includes an ultrasonic transducer configured to transmit ultrasound waves at a first frequency and a second frequency through a skull of the subject toward brain tissue and cerebrospinal fluid within the skull. The system includes a receiver configured to detect a parametric array signal generated by nonlinear interaction of the ultrasound waves at the first and second frequencies within the brain tissue and cerebrospinal fluid, wherein the parametric array signal includes a difference frequency component corresponding to a difference between the first frequency and the second frequency. The system includes a processor configured to determine a change in a ratio of cerebrospinal fluid to brain tissue based on a magnitude of the parametric array signal.

### CROSS-REFERENCE TO RELATED APPLICATIONS

This application claims the benefit of U.S. Provisional Application Ser. No. 63/806,223, filed on 15 May 2025, which is incorporated herein by reference in its entirety as if fully set forth below.

### GOVERNMENT LICENSE RIGHTS

This invention was made with government support under 1933158, and 1966158 awarded by the National Science Foundation. The government has certain rights in this invention.

### FIELD OF INVENTION

The present disclosure relates to medical imaging and diagnostic systems, and more particularly to a noninvasive ultrasound-based method for monitoring changes in intracranial pressure and cerebrospinal fluid volume in hydrocephalus patients by exploiting parametric array signals and differing nonlinear acoustic properties of brain tissue versus cerebrospinal fluid.

### BACKGROUND

Hydrocephalus is a neurological condition characterized by excessive accumulation of cerebrospinal fluid within the ventricles of the brain, leading to elevated intracranial pressure. The management of hydrocephalus involves frequent monitoring of intracranial pressure, which serves as a biomarker for tracking disease progression and guiding treatment decisions regarding the removal of excess cerebrospinal fluid. The mainstay of treatment often involves implantation of a shunt system that drains excess cerebrospinal fluid away from the brain, thus alleviating elevated intracranial pressure and associated symptoms. Invasive intracranial pressure monitoring using external ventricular drains or parenchymal microsensors represents the gold standard in clinical practice. Non-invasive surrogates such as Transcranial Doppler ultrasonography or Optic Nerve Sheath Diameter measurements have also been developed to assess intracranial conditions.

However, existing approaches for monitoring intracranial pressure and cerebrospinal fluid dynamics suffer from several limitations. Invasive catheter-based methods inserted directly into the ventricles or brain parenchyma carry risks such as infection, hemorrhage, mechanical failure, and neurological damage. Such invasive monitoring is also episodic, and clinicians often lack continuous insight into intracranial dynamics in the outpatient setting after catheter removal. Non-invasive surrogates such as Transcranial Doppler ultrasonography often lack a direct physical correlation with ventricular volume and result in operator variability, as these are indirect measurements often confounded by systemic hemodynamics. Linear pulse-echo ultrasound techniques that track the geometric expansion of the ventricles by measuring the distance to the brain-water interface rely on precise registration to capture specular reflections from the ventricular walls, making them operator-dependent and prone to signal loss due to skull-induced scattering or misalignment.

What is needed, therefore, is an improved system and method for noninvasive monitoring of intracranial conditions that can accurately detect ventricular volume changes and reliably assess shunt function while reducing complications associated with invasive monitoring techniques. Such a system would address the monitoring gap that exists once a patient leaves the hospital and provide continuous or frequent monitoring capabilities in outpatient settings.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

A system for monitoring intracranial conditions in a subject can include an ultrasonic transducer configured to transmit ultrasound waves at a first frequency and a second frequency through a skull of the subject toward brain tissue and cerebrospinal fluid within the skull. The system can include a receiver configured to detect a parametric array signal generated by nonlinear interaction of the ultrasound waves at the first frequency and the second frequency within the brain tissue and the cerebrospinal fluid. The parametric array signal can include a difference frequency component corresponding to a difference between the first frequency and the second frequency. The system can include a processor configured to determine a change in a ratio of the cerebrospinal fluid to the brain tissue based on a magnitude of the parametric array signal.

A method for monitoring intracranial conditions in a subject can include transmitting, via an ultrasonic transducer, ultrasound waves at a first frequency and a second frequency through a skull of the subject toward brain tissue and cerebrospinal fluid within the skull. The method can include detecting, via a receiver, a parametric array signal generated by nonlinear interaction of the ultrasound waves at the first frequency and the second frequency within the brain tissue and the cerebrospinal fluid. The parametric array signal can include a difference frequency component corresponding to a difference between the first frequency and the second frequency. The method can include determining, via a processor, a change in a ratio of the cerebrospinal fluid to the brain tissue based on a magnitude of the parametric array signal.

A portable device for noninvasive monitoring of cerebrospinal fluid dynamics in a subject can include an ultrasonic transducer configured to transmit ultrasound waves at a first frequency and a second frequency through a skull of the subject. The portable device can include a receiver configured to detect a parametric array signal at a difference frequency generated by nonlinear interaction of the ultrasound waves within brain tissue and cerebrospinal fluid of the subject. The portable device can include a signal processing unit configured to analyze the parametric array signal to determine changes in a ratio of the cerebrospinal fluid to the brain tissue. The portable device can include a user interface configured to display data indicative of changes in ventricular size based on the parametric array signal.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 illustrates a system for monitoring intracranial pressure in hydrocephalus having an ultrasonic transducer, according to examples of the disclosed technology.

FIG. 2 illustrates a graph showing a relationship between a parametric array signal magnitude and frequency, according to examples of the disclosed technology.

FIG. 3 illustrates a comparison of primary frequency pressure distribution and parametric array difference frequency pressure distribution, according to examples of the disclosed technology.

FIG. 4 illustrates simulation results showing ventricular expansion detection using parametric acoustic feedback, according to examples of the disclosed technology.

FIG. 5 illustrates a simulation mask showing a cross-sectional view of a skull with speed of sound values, according to examples of the disclosed technology.

FIG. 6 illustrates a nonlinearity parameter map of brain tissue and cerebrospinal fluid within a skull cavity, according to examples of the disclosed technology.

FIG. 7 illustrates a graph showing a relationship between normalized level and ventricular cross-sectional area, according to examples of the disclosed technology.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

The present disclosure relates to systems and methods for noninvasive monitoring of intracranial conditions, and more particularly to ultrasound-based techniques that leverage parametric array signals to detect changes in cerebrospinal fluid volume and infer variations in intracranial pressure. The disclosed technology can provide continuous or frequent monitoring capabilities for hydrocephalus patients in both clinical and outpatient settings, addressing limitations associated with invasive catheter-based monitoring approaches.

Hydrocephalus management involves tracking changes in intracranial pressure as a biomarker for disease progression and treatment efficacy. Removal of excess cerebrospinal fluid through shunt-based treatment can lead to a reduction in intracranial pressure and a corresponding increase in the space occupied by brain tissue within the skull. The disclosed technology exploits the differing nonlinear acoustic properties of brain tissue and cerebrospinal fluid to detect relative changes in ventricular volume. Brain tissue and cerebrospinal fluid possess distinct acoustic nonlinearity parameters, and this contrast can be leveraged to monitor shifts in the ratio of cerebrospinal fluid to brain tissue within the intracranial space.

The disclosed systems and methods can utilize parametric array signals generated by nonlinear interaction of high-frequency ultrasound waves propagating through the skull and into the brain. When two primary ultrasound waves at different frequencies interact within a nonlinear medium, secondary low-frequency waves can be generated at a difference frequency. The magnitude of these parametric array signals can depend on the effective nonlinearity parameter of the tissue through which the ultrasound waves propagate. As ventricular volume changes due to accumulation or drainage of cerebrospinal fluid, the effective nonlinearity parameter within the acoustic interaction zone can shift, resulting in detectable changes in the parametric array signal magnitude.

The disclosed technology can provide several advantages for monitoring intracranial conditions. Noninvasive monitoring can reduce risks associated with invasive intracranial pressure measurement techniques, such as infection, hemorrhage, and neurological damage. The disclosed systems can avoid the use of contrast agents by leveraging intrinsic acoustic properties of brain tissue and cerebrospinal fluid. Portable implementations of the disclosed technology can be suitable for point-of-care and remote settings, facilitating frequent measurements without subjecting patients to invasive procedures. The disclosed systems can also provide real-time feedback on intracranial pressure status and shunt functionality, enabling clinicians to assess treatment efficacy and detect changes in ventricular size over time.

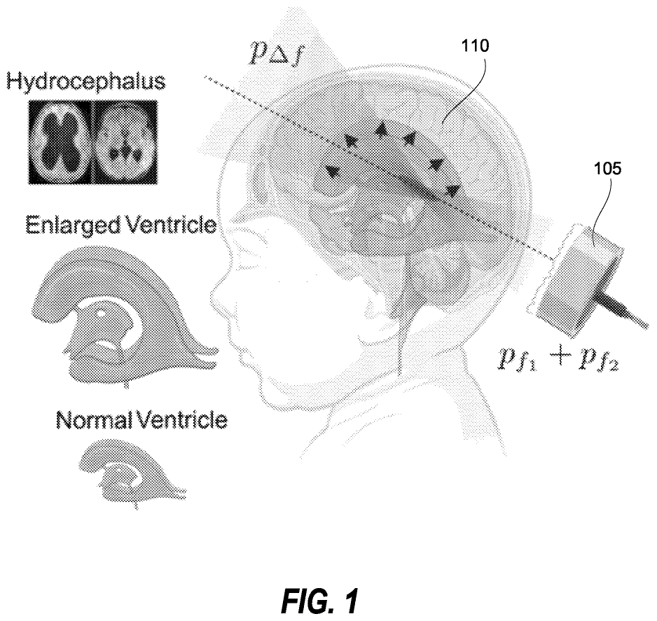

FIG. 1 illustrates a system for monitoring intracranial conditions in a subject. The system can include an ultrasonic transducer 110 configured to transmit ultrasound waves at a first frequency and a second frequency through a skull of the subject toward brain tissue and cerebrospinal fluid within the skull. The ultrasonic transducer 110 can be positioned adjacent to a parietal region of the skull and can generate primary high-frequency ultrasound beams that propagate through the skull and into the brain. The ultrasonic transducer 110 can generate a parametric array signal through nonlinear mixing of high-frequency waves within the propagation medium. When two primary ultrasound waves at different frequencies interact within a nonlinear medium such as brain tissue and cerebrospinal fluid, secondary low-frequency waves can be generated at a difference frequency.

The system can include a receiver 105 configured to detect a parametric array signal generated by nonlinear interaction of the ultrasound waves at the first frequency and the second frequency within the brain tissue and the cerebrospinal fluid. The parametric array signal can include a difference frequency component corresponding to a difference between the first frequency and the second frequency. The receiver 105 can be positioned on a distal side of the skull from the ultrasonic transducer 110 to detect the parametric array signal that has propagated through the skull, brain tissue, ventricles, and cerebrospinal fluid. The ultrasonic transducer 110 and the receiver 105 can be configured in a through-transmission arrangement where the ultrasound waves pass completely through the skull from one side to the other. In some cases, the first frequency and the second frequency can be in a megahertz range, and the difference frequency component can be in a kilohertz range. For example, primary frequencies of 0.95 MHz and 1.05 MHz can result in a 100 kHz difference frequency. The lower difference frequency can penetrate through the skull with reduced attenuation compared to the primary high-frequency waves.

The system can include a processor configured to determine a change in a ratio of the cerebrospinal fluid to the brain tissue based on a magnitude of the parametric array signal. The processor can be further configured to correlate the change in the ratio of the cerebrospinal fluid to the brain tissue with a change in ventricular size within the skull. The processor can be further configured to infer a change in intracranial pressure based on the change in ventricular size. Brain tissue and cerebrospinal fluid possess distinct acoustic nonlinearity parameters, with brain tissue having a nonlinearity parameter of approximately 4.7 and cerebrospinal fluid having a nonlinearity parameter of approximately 3.6. As ventricular volume changes due to accumulation or drainage of cerebrospinal fluid, the effective nonlinearity parameter within the acoustic interaction zone can shift, resulting in detectable changes in the parametric array signal magnitude.

The ultrasonic transducer 110 can have a configurable F-number to adjust a focus of the ultrasound waves based on anatomy of the subject. The configurable F-number can allow users to adjust focus tightness based on patient-specific anatomy or clinical application. The F-number can be configured by adjusting the focal length of the holographic lens relative to the aperture diameter of the ultrasonic transducer 110. In some cases, the system can include interchangeable holographic lenses having different focal lengths to provide different F-number configurations. In some cases, the ultrasonic transducer 110 can include an adjustable aperture to modify the effective aperture diameter. The system can further include a signal processing unit configured to demodulate and filter the parametric array signal received by the receiver 105. The signal processing unit can include a processing platform with software and hardware that demodulates and filters the received parametric array signals. Advanced beamforming and nonlinear acoustic models can correlate changes in parametric array signal amplitude with local brain tissue and cerebrospinal fluid properties.

The ultrasonic transducer 110 can include a holographic lens configured to focus the ultrasound waves through the skull. The ultrasonic transducer 110 can be coupled to a 3D-printed holographic lens that focuses the ultrasound beam at two different frequencies. The holographic lens can include a phase-engineered lens configured to compensate for skull-induced aberrations. The holographic lens can be designed using a topology optimization approach that accounts for skull-induced aberrations through heterogeneous angular spectrum methods. The holographic lens can be designed using a framework that combines the Heterogeneous Angular Spectrum Approach (HASA) with an iterative optimizer such as ADAM to optimize lens thickness profiles. The framework can account for in-plane varying speed-of-sound maps and support differentiable optimization of lens thickness profiles. The phase patterns can be encoded by varying the physical thickness of the lens material across the aperture, where the thickness at each location determines the local phase shift imparted to the ultrasound wave. The holographic lens can include a phase plate that encodes spatial phase patterns to transform a single-element transducer into a device capable of generating complex volumetric pressure fields. By calculating a phase map using time-reversal or similar algorithms, the holographic lens can be tailored to an individual patient's skull and thereby correct specific aberrations without complex active electronics associated with phased arrays.

The processor can be further configured to establish a baseline parametric array signal magnitude and compare a subsequently measured parametric array signal magnitude to the baseline parametric array signal magnitude to detect a change in ventricular size. A baseline scan can be performed to map the patient's typical acoustic properties and establish a reference parametric array signal. The baseline can measure expected ventricular geometry and the associated parametric array signal level. Periodic or continuous scans can be acquired during hydrocephalus progression or post-shunt placement. A decrease in the parametric array signal magnitude relative to the baseline parametric array signal magnitude can indicate ventricular expansion. As ventricles expand during untreated hydrocephalus, the parametric array signal can drop in intensity. An increase in the parametric array signal magnitude relative to the baseline parametric array signal magnitude can indicate ventricular contraction. As ventricles contract during functional shunt drainage, the parametric array signal can intensify relative to baseline.

The processor can be further configured to assess functionality of a hydrocephalus shunt based on the magnitude of the parametric array signal. The system can provide a noninvasive, real-time approach to confirming shunt efficacy, reducing infection and hemorrhage risks associated with invasive monitoring techniques. The system can be configured as a wearable helmet-like device that the patient can use at home for continuous monitoring. The ultrasonic transducer 110 and holographic lens assembly can be designed to be portable for point-of-care and remote settings. The system can be configured for pediatric use, leveraging natural acoustic windows such as fontanelles for improved imaging in infants.

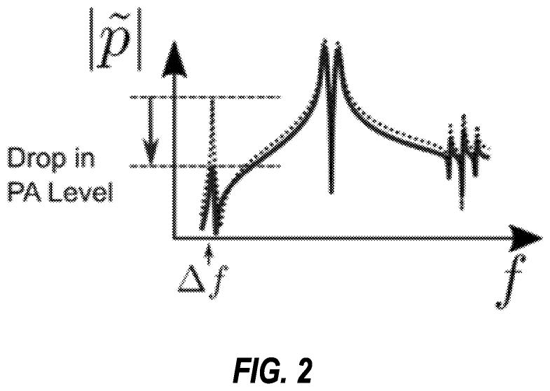

FIG. 2 illustrates a graph showing a relationship between parametric array signal magnitude and frequency for monitoring changes in ventricular size associated with hydrocephalus. The vertical axis of the graph represents the absolute value of the parametric pressure signal, while the horizontal axis represents frequency. A difference frequency is indicated along the horizontal axis. The graph shows a spectral response curve with a pronounced dip at a specific frequency region, where the drop in parametric array level is annotated. Two overlapping curves are presented, one shown as a solid line and one shown as a dotted line, representing different ventricular states or conditions. The drop in parametric array level corresponds to the change in signal magnitude that occurs when ventricular volume increases due to accumulation of cerebrospinal fluid, as the nonlinear acoustic properties of brain tissue differ from those of cerebrospinal fluid.

As described above, the first frequency and the second frequency can be in a megahertz range, and the difference frequency component can be in a kilohertz range. The ultrasonic transducer can transmit primary frequencies in the megahertz range, such as around 1 MHz and 1.05 MHz, to generate a difference frequency of approximately 50 kHz. In some cases, primary frequencies of 0.95 MHz and 1.05 MHz can be emitted by a focused transducer to generate a 100 kHz difference frequency. The lower difference frequency can penetrate through the skull with reduced attenuation compared to the primary high-frequency waves. The primary beam at approximately 1 MHz can attenuate heavily after passing through the focal plane and crossing the distal skull layer, while the 100 kHz difference frequency can cross the distal skull with negligible loss due to favorable inverse-frequency attenuation characteristics.

The difference frequency component can range from 50 kHz to 175 kHz for contrast between normal and diseased ventricular states. At low difference frequencies (less than or equal to 50 kHz), the nonlinear conversion efficiency can drop as expected from the theory of parametric acoustic arrays, and larger acoustic wavelengths (approximately 30 mm) can lead to diffraction, resulting in a diffuse beam that fails to maintain effective collimation to interact with the ventricles. Generating higher difference frequencies (such as 200 kHz) can require a wider separation between the primary frequencies, which can be limited by the bandwidth of piezoelectric transducers. A larger separation can reduce primary pressure amplitudes. The 75 kHz to 125 kHz range can serve as an operational window where the parametric conversion efficiency is sufficient to generate a measurable signal and the beam maintains effective collimation to interact with the ventricles. In this regime, the diagnostic contrast can remain stable at approximately 10 to 15 percent.

The difference frequency can be approximately 10% of the primary frequency to achieve maximum signal drop for detecting ventricular changes. For example, if the primary frequency is 1 MHz, a difference frequency of around 100 kHz can provide maximum drop in the parametric array signal. The relationship between the parametric array signal amplitude and downshift ratio (primary frequency divided by difference frequency) follows established parametric array theory, where larger downshift ratios yield smaller parametric array signals owing to lower nonlinear interaction efficiency. Conversely, smaller downshift ratios, while producing stronger signals, can require transducers with wider bandwidth capabilities. The frequency at which the difference frequency component is measured provides information about the relative changes in ventricular size, with the signal magnitude decreasing as the ventricles expand during hydrocephalus progression.

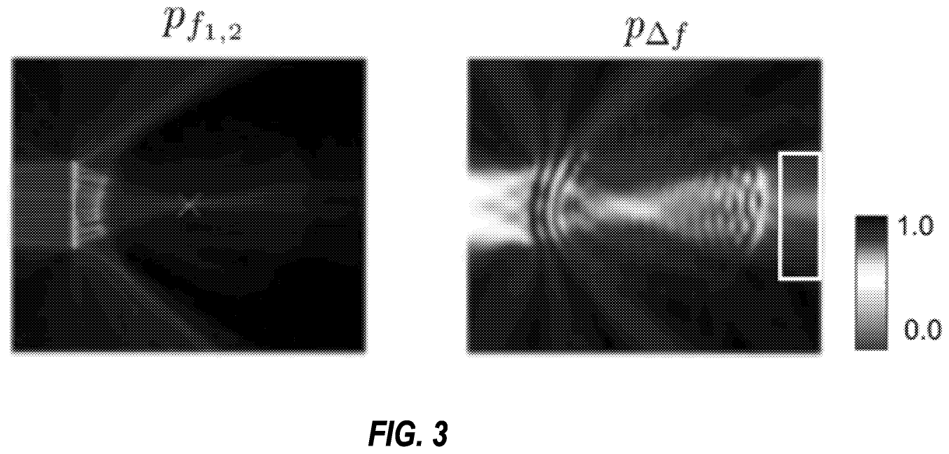

FIG. 3 illustrates a comparison of acoustic pressure field images showing primary frequency pressure distribution and parametric array difference frequency pressure distribution during transcranial ultrasound propagation. The figure presents two side-by-side images that demonstrate the distinct propagation characteristics of the primary high-frequency waves and the secondary low-frequency parametric array signal.

The left image of FIG. 3 shows the pressure field at the primary frequencies, labeled as p_{f1,2}, displaying a focused beam pattern that converges toward a central focal point and then diverges. The highest intensity of the primary frequency pressure field can be concentrated in a narrow region near the focal zone. The focused beam pattern demonstrates the convergence of the ultrasound waves as the waves propagate through the skull and into the brain tissue.

The right image of FIG. 3 shows the parametric array pressure field at the difference frequency, labeled as p_{Δf}, displaying a broader spatial distribution of acoustic energy that extends further into the propagation medium beyond the focal region. The parametric array pressure field can exhibit a more diffuse spatial profile compared to the primary frequency pressure field, with the acoustic energy distributed over a larger volume as the difference frequency signal propagates through the intracranial space.

A grayscale intensity bar can be positioned adjacent to the images, indicating normalized pressure values ranging from 0.0 to 1.0. Lighter shades can represent higher pressure amplitudes and darker shades can represent lower pressure amplitudes. The normalized pressure values can provide a quantitative reference for comparing the relative intensities of the primary frequency and difference frequency pressure fields.

The comparison between the two images illustrates how the primary high-frequency waves attenuate more rapidly after passing through the skull and brain tissue. The primary beam at approximately 1 MHz can suffer approximately 20 dB attenuation and scattering at the entry skull interface. After passing through the focal plane and crossing the distal skull layer, the primary high-frequency waves can attenuate heavily due to frequency-dependent absorption characteristics of bone and soft tissue.

In contrast, the lower difference frequency signal generated through nonlinear wave mixing can propagate with less attenuation through the intracranial medium. The difference frequency signal at approximately 100 kHz can cross the distal skull with negligible loss due to favorable inverse-frequency attenuation characteristics. The skull can act as a low-pass filter that isolates the diagnostic tone, allowing the parametric array signal to emerge on the distal side of the skull with detectable amplitude.

The difference frequency can be generated cumulatively along the propagation path within the tissue. This cumulative generation can allow the parametric array signal to maintain a coherent beam profile that effectively tunnels through the distal skull layer. A receiver positioned on an opposite side of the skull from the ultrasonic transducer can detect the low-frequency parametric array signal that survives attenuation through the skull due to the lower frequency of the parametric array signal compared to the primary frequencies. The through-transmission arrangement can enable detection of the parametric array signal in transmission mode outside the skull cavity, where the signal carries information about the acoustic properties of the brain tissue and cerebrospinal fluid through which the ultrasound waves have propagated.

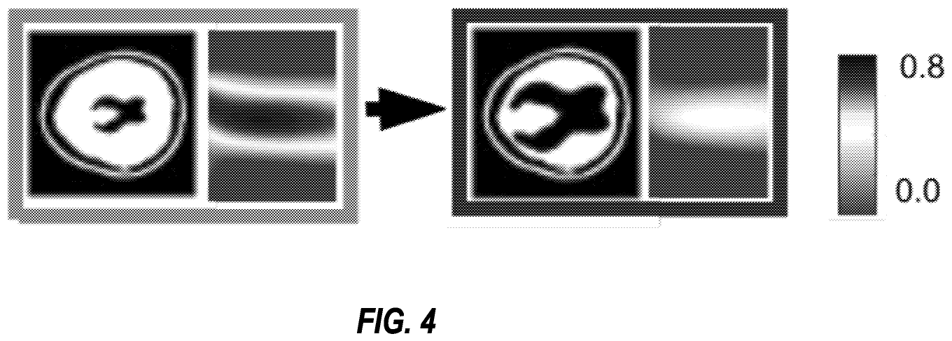

FIG. 4 illustrates simulation results showing ventricular expansion detection using parametric acoustic feedback. The figure presents two panels arranged horizontally with an arrow pointing from the left panel to the right panel, indicating a transition or progression from a baseline ventricular state to an enlarged ventricular state associated with hydrocephalus progression.

Each panel of FIG. 4 contains two side-by-side images. The left image in each panel shows a circular cross-sectional view of a skull with an irregularly shaped dark region in the center representing the ventricles. The right image in each panel displays a grayscale gradient pattern representing acoustic field intensity. A grayscale colorbar can be positioned to the right of the figure, with normalized intensity values ranging from 0.0 at the bottom to 0.8 at the top.

The left panel of FIG. 4 represents a baseline or normal ventricular state. The dark ventricular region in the left panel can correspond to a ventricular cross-sectional area of approximately 20 cm², representing moderate ventriculomegaly. The corresponding acoustic field pattern in the right image of the left panel shows the parametric array signal intensity distribution for the baseline ventricular state.

The right panel of FIG. 4 represents an enlarged ventricular state associated with hydrocephalus progression. The dark ventricular region in the right panel appears larger than in the left panel, demonstrating the expansion of the ventricles. The enlarged ventricular region can correspond to a ventricular cross-sectional area of approximately 40 cm², representing a doubling of the ventricular size from the baseline state.

The corresponding acoustic field patterns in the right images of each panel show differences in intensity distribution, reflecting the change in parametric array signal generation due to the altered ratio of brain tissue to cerebrospinal fluid as the ventricles expand. As the ventricular volume increases, the effective nonlinearity parameter within the acoustic interaction zone can shift. Brain tissue can have a nonlinearity parameter of approximately 4.7, while cerebrospinal fluid can have a nonlinearity parameter of approximately 3.5 to 3.6. The expanding ventricles can replace higher-nonlinearity brain tissue with lower-nonlinearity cerebrospinal fluid in the interaction zone.

The simulation results demonstrate that as the ventricular cross-sectional area increases from approximately 20 cm² to approximately 40 cm², the parametric array signal measured outside the skull cavity can drop by approximately 10 percent. This drop in amplitude can indicate a decrease in nonlinear generation efficiency, as the interaction zone becomes increasingly occupied by low-nonlinearity cerebrospinal fluid. The decrease in parametric array signal magnitude can also be sensitive to the transducer F-number, where tightly focused beams can lead to a larger relative drop in parametric pressure.

The simulation results shown in FIG. 4 support the use of parametric array signals for tracking relative changes in ventricular size associated with hydrocephalus progression or treatment response. The contrast between the normal and hydrocephalus states can provide a diagnostic margin for reliable differentiation between the two states. A parametric array signal below a baseline threshold can indicate pathological ventricular expansion without requiring complex image reconstruction or expert operator interpretation.

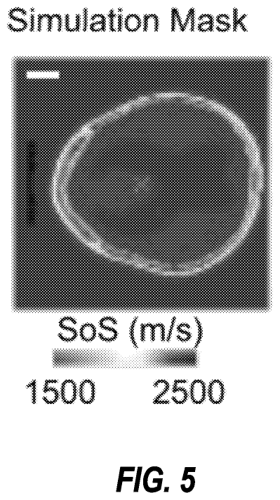

FIG. 5 illustrates a simulation mask showing a cross-sectional view of a skull with associated speed of sound values for transcranial ultrasound monitoring. The simulation mask can be used to model acoustic wave propagation through the cranium for hydrocephalus monitoring applications.

The simulation mask displays an approximately circular skull boundary rendered in lighter tones against a dark background. The interior region of the skull can be shown in darker shades representing different tissue types, including brain tissue and ventricular spaces. The skull boundary can exhibit an irregular shape that represents anatomical features of the cranium.

A scale bar can be positioned in the upper left corner of the image to indicate spatial dimensions. The speed of sound (“SoS”) scale, labeled as SoS (m/s), can be provided below the image and can range from 1500 m/s to 2500 m/s. The gradient bar can transition from dark to light corresponding to increasing speed of sound values.

The skull boundary can exhibit higher speed of sound values as indicated by the lighter coloration. Cortical bone of the skull can have a speed of sound of approximately 2500 m/s. The interior regions including brain tissue and ventricular spaces can display lower speed of sound values represented by darker shading. Water and soft tissue can have a speed of sound of approximately 1480 m/s to 1500 m/s. The speed of sound can discontinuously jump from approximately 1480 m/s to over 2500 m/s within a sub-millimeter distance at the water-skull boundary.

The simulation mask can be derived from micro-CT scan data of a human skull. The three-dimensional geometry can be acquired via a micro-CT scan and interpolated onto a voxel grid. Speed-of-sound and density maps can be obtained from the micro-CT data and used to create spatially varying acoustic property maps. The density and longitudinal speed of sound for each voxel can be linearly interpolated between baseline properties of pure water and maximum properties of solid bone.

The heterogeneous acoustic property map represented by the simulation mask can be used to perform patient-specific transcranial focused ultrasound simulations. The spatially varying speed-of-sound maps can capture refraction and transmission of waves through the skull and into the brain tissue. The simulation mask can account for the strong heterogeneity of the skull, where the speed of sound varies across different tissue types and anatomical structures.

The contrast in speed of sound values between the skull boundary and interior regions can affect how ultrasound waves propagate through the cranium. The higher speed of sound in the skull bone compared to the surrounding soft tissue and cerebrospinal fluid can cause refraction of acoustic waves at tissue interfaces. The simulation mask can be used to model these acoustic interactions and predict how parametric array signals propagate through the intracranial space for hydrocephalus monitoring.

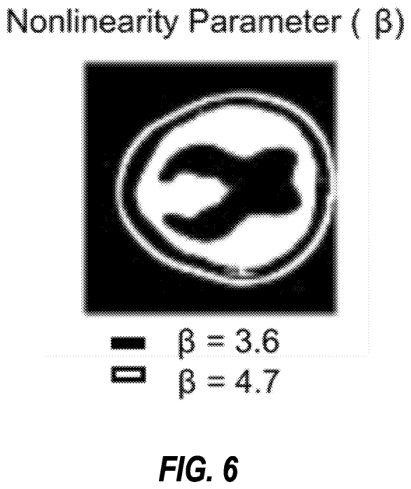

FIG. 6 illustrates a nonlinearity parameter map of brain tissue and cerebrospinal fluid within a skull cavity. The nonlinearity parameter map can be used in conjunction with the simulation mask described above to model parametric array signal generation for hydrocephalus monitoring applications.

The nonlinearity parameter map displays a cross-sectional image showing a circular region representing the skull boundary. Within the skull boundary, irregular dark regions can indicate areas of differing acoustic nonlinearity corresponding to brain ventricles and surrounding brain tissue. The ventricular spaces can appear as distinct regions within the cross-sectional view, with the shape and size of the ventricular regions varying based on the degree of ventricular expansion associated with hydrocephalus.

A legend can be provided below the image indicating two distinct nonlinearity parameter values. A filled rectangle can correspond to a nonlinearity parameter value of β≈3.6 representing cerebrospinal fluid. An unfilled rectangle can correspond to a nonlinearity parameter value of β≈4.7 representing brain tissue. In some cases, literature values for the nonlinearity parameter can vary, with cerebrospinal fluid having a nonlinearity parameter of approximately β≈5.2 and brain tissue having a nonlinearity parameter of approximately β≈6.6. The nonlinearity parameter β can be referred to herein as the acoustic nonlinearity parameter.

The contrast between the nonlinearity parameter values of brain tissue and cerebrospinal fluid forms the basis for detecting changes in ventricular size associated with hydrocephalus. Brain tissue can possess a higher nonlinearity parameter compared to cerebrospinal fluid. When ultrasound waves propagate through the intracranial space and interact nonlinearly within the brain tissue and cerebrospinal fluid, the magnitude of the parametric array signal generated at the difference frequency can depend on the effective nonlinearity parameter of the medium through which the ultrasound waves propagate.

As ventricular volume increases during hydrocephalus progression, higher-nonlinearity brain tissue can be replaced by lower-nonlinearity cerebrospinal fluid within the acoustic interaction zone. This volumetric replacement can cause a reduction in the effective nonlinearity parameter within the focal zone, resulting in decreased nonlinear generation efficiency. The decrease in nonlinear generation efficiency can manifest as a drop in the parametric array signal magnitude measured outside the skull cavity.

Conversely, as ventricular volume decreases during successful shunt treatment, lower-nonlinearity cerebrospinal fluid can be replaced by higher-nonlinearity brain tissue within the acoustic interaction zone. This volumetric replacement can cause an increase in the effective nonlinearity parameter within the focal zone, resulting in increased nonlinear generation efficiency. The increase in nonlinear generation efficiency can manifest as a rise in the parametric array signal magnitude measured outside the skull cavity.

The nonlinearity parameter map can be used in simulations to predict how changes in the ratio of cerebrospinal fluid to brain tissue affect the parametric array signal. By assigning the appropriate nonlinearity parameter values to the ventricular regions and surrounding brain tissue, the simulation can model the nonlinear acoustic interactions that occur as ultrasound waves propagate through the intracranial space. The simulation results can be used to establish relationships between ventricular size changes and corresponding changes in parametric array signal magnitude for hydrocephalus monitoring applications.

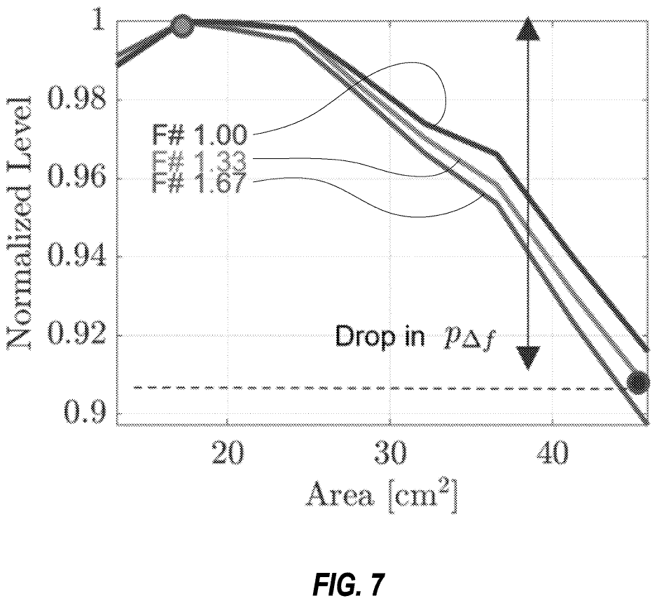

FIG. 7 illustrates a graph showing a relationship between normalized level and ventricular cross-sectional area for monitoring intracranial pressure changes in hydrocephalus. The graph can be used to quantify the relationship between ventricular expansion and parametric array signal magnitude.

The vertical axis of the graph represents normalized level ranging from approximately 0.9 to 1.0. The normalized level can correspond to the parametric array signal magnitude normalized to a baseline value. The horizontal axis represents ventricular cross-sectional area measured in square centimeters, ranging from approximately 20 cm² to 40 cm². The ventricular cross-sectional area can represent the size of the ventricles as viewed in a cross-sectional plane through the skull.

Three curves are shown in the graph corresponding to different F-number values of 1.00, 1.33, and 1.67. Each curve can be represented by a distinct line style. The F-number can correspond to the ratio of focal length to aperture diameter of the ultrasonic transducer. Lower F-number values can correspond to more tightly focused beams, while higher F-number values can correspond to more weakly focused beams.

The curves demonstrate a general downward trend as the ventricular cross-sectional area increases. All three curves can start near a normalized level of 1.0 at approximately 20 cm² and can decrease as the area expands toward 40 cm². The downward trend can reflect the decrease in parametric array signal magnitude that occurs as the ventricles expand and higher-nonlinearity brain tissue is replaced by lower-nonlinearity cerebrospinal fluid within the acoustic interaction zone.

A horizontal dashed line can be positioned at approximately 0.905 on the normalized level axis, serving as a reference threshold. The reference threshold can represent a diagnostic boundary for distinguishing between normal and pathological ventricular states. A vertical double-headed arrow labeled as the drop in parametric array signal pressure can indicate the magnitude of signal decrease between the initial normalized level of 1.0 and the dashed reference line.

The parametric array signal magnitude can drop by approximately 10 percent when the ventricular cross-sectional area increases from 20 cm² to 40 cm². This drop in amplitude can indicate a decrease in nonlinear generation efficiency, as the interaction zone becomes increasingly occupied by low-nonlinearity cerebrospinal fluid. The decrease in parametric array signal magnitude can also be sensitive to the transducer F-number. Tightly focused beams corresponding to lower F-number values such as 1.00 can lead to a larger relative drop in parametric pressure compared to more weakly focused beams corresponding to higher F-number values such as 1.67.

The graph supports the use of parametric array signals for tracking relative changes in ventricular size associated with hydrocephalus progression or treatment response. The contrast between the baseline normalized level and the reduced normalized level at larger ventricular cross-sectional areas can provide a diagnostic margin for reliable differentiation between normal and hydrocephalus states. A parametric array signal below the reference threshold can indicate pathological ventricular expansion.

A method for monitoring intracranial conditions in a subject can include transmitting, via an ultrasonic transducer, ultrasound waves at a first frequency and a second frequency through a skull of the subject toward brain tissue and cerebrospinal fluid within the skull. The ultrasonic transducer can be positioned adjacent to the skull and configured to generate primary high-frequency ultrasound beams that propagate through the skull and into the brain. The primary frequencies transmitted by the ultrasonic transducer can range from 50 kHz to 20 MHz depending on the application and tissue being monitored. The ultrasonic transducer can be configured to operate at lower primary frequencies when imaging through the skull to reduce attenuation, and at higher frequencies when imaging less lossy tissues such as the abdomen.

The method can include detecting, via a receiver, a parametric array signal generated by nonlinear interaction of the ultrasound waves at the first frequency and the second frequency within the brain tissue and the cerebrospinal fluid. The parametric array signal can include a difference frequency component corresponding to a difference between the first frequency and the second frequency. The parametric array signal can be generated when the two primary ultrasound waves at different frequencies interact within a nonlinear medium such as brain tissue and cerebrospinal fluid, producing secondary low-frequency waves at the difference frequency.

The method can include determining, via a processor, a change in a ratio of the cerebrospinal fluid to the brain tissue based on a magnitude of the parametric array signal. The processor can analyze the parametric array signal magnitude to detect shifts in the effective nonlinearity parameter of the tissue through which the ultrasound waves have propagated. Brain tissue and cerebrospinal fluid possess distinct acoustic nonlinearity parameters, and changes in the ratio of cerebrospinal fluid to brain tissue within the intracranial space can result in detectable changes in the parametric array signal magnitude.

The method can include establishing a baseline parametric array signal magnitude. A baseline scan can be performed to map the acoustic properties of the subject and establish a reference parametric array signal. The baseline can measure expected ventricular geometry and the associated parametric array signal level in a healthy or stable state.

The method can include comparing a subsequently measured parametric array signal magnitude to the baseline parametric array signal magnitude to infer a change in ventricular size. Periodic or continuous scans can be acquired during hydrocephalus progression or post-shunt placement. Deviations in the measured parametric array signal magnitude relative to the baseline can indicate changes in ventricular size. A decrease in the parametric array signal magnitude relative to the baseline parametric array signal magnitude can indicate ventricular expansion, as the expanding ventricles replace higher-nonlinearity brain tissue with lower-nonlinearity cerebrospinal fluid. An increase in the parametric array signal magnitude relative to the baseline parametric array signal magnitude can indicate ventricular contraction, as successful shunt drainage replaces lower-nonlinearity cerebrospinal fluid with higher-nonlinearity brain tissue.

The method can include inferring a change in intracranial pressure based on the change in ventricular size. Changes in ventricular size can correlate with changes in intracranial pressure, as removal of excess cerebrospinal fluid through shunt-based treatment can lead to a reduction in intracranial pressure and a corresponding decrease in ventricular volume. The processor can infer the change in intracranial pressure by correlating the change in ventricular size with established clinical relationships between ventricular volume and intracranial pressure. The processor can apply a lookup table, a mathematical model, or a machine learning algorithm trained on clinical data to map changes in ventricular size to corresponding changes in intracranial pressure.

Transmitting the ultrasound waves can include focusing the ultrasound waves through the skull via a holographic lens coupled to the ultrasonic transducer. The holographic lens can focus and direct the ultrasound beam through the skull with reduced distortion. The holographic lens can include a phase-engineered lens configured to compensate for skull-induced aberrations. The holographic lens can be designed using a topology optimization approach that accounts for skull-induced aberrations through heterogeneous angular spectrum methods.

Detecting the parametric array signal can include receiving the parametric array signal at a location on an opposite side of the skull from the ultrasonic transducer. The receiver can be positioned on a distal side of the skull from the ultrasonic transducer in a through-transmission arrangement. The lower difference frequency can penetrate through the skull with reduced attenuation compared to the primary high-frequency waves, allowing the parametric array signal to be detected in transmission mode outside the skull cavity.

The method can include continuously monitoring the magnitude of the parametric array signal over a period of time to track changes in ventricular size. The continuous monitoring can provide a data refresh rate of approximately 1 Hz, generating one data point every second. The pulses can be delivered at a pulse repetition frequency of 1 to 2 Hz as per the desired sensor refresh rate. Each pulse can include 30 to 40 cycles of the primary frequency, resulting in a pulse duration of approximately 30 to 40 microseconds for a 1 MHz primary frequency. The pulse parameters can be configured to generate parametric array signals without crossing thermal and mechanical safety thresholds. The continuous monitoring can enable tracking of ventricular changes over time, providing clinicians with real-time feedback on intracranial pressure status and shunt functionality.

A portable device for noninvasive monitoring of cerebrospinal fluid dynamics in a subject can include an ultrasonic transducer configured to transmit ultrasound waves at a first frequency and a second frequency through a skull of the subject. The ultrasonic transducer can be configured to generate primary high-frequency ultrasound beams that propagate through the skull and into the brain tissue and cerebrospinal fluid. The ultrasonic transducer can include a holographic lens configured to focus the ultrasound waves through the skull. The holographic lens can include a phase-engineered lens configured to compensate for skull-induced aberrations. The portable device can be housed in a compact form factor suitable for point-of-care and remote settings. In some cases, the portable device can be configured as a wearable helmet-like device that a patient can use at home for continuous monitoring.

The portable device can include a receiver configured to detect a parametric array signal at a difference frequency generated by nonlinear interaction of the ultrasound waves within brain tissue and cerebrospinal fluid of the subject. The parametric array signal can be generated when the two primary ultrasound waves at different frequencies interact within a nonlinear medium such as brain tissue and cerebrospinal fluid, producing secondary low-frequency waves at the difference frequency. The receiver can be positioned on an opposite side of the skull from the ultrasonic transducer in a through-transmission arrangement. The lower difference frequency can penetrate through the skull with reduced attenuation compared to the primary high-frequency waves, allowing the parametric array signal to be detected in transmission mode outside the skull cavity.

The portable device can include a signal processing unit configured to analyze the parametric array signal to determine changes in a ratio of the cerebrospinal fluid to the brain tissue. The signal processing unit can include a processing platform with software and hardware that demodulates and filters the received parametric array signals. The signal processing unit can employ beamforming and nonlinear acoustic models to correlate changes in parametric array signal amplitude with local brain tissue and cerebrospinal fluid properties. The signal processing unit can be configured to establish a baseline parametric array signal magnitude and compare subsequently measured parametric array signal magnitudes to the baseline parametric array signal magnitude to detect changes in ventricular size. A decrease in the parametric array signal magnitude relative to the baseline parametric array signal magnitude can indicate ventricular expansion, as the expanding ventricles replace higher-nonlinearity brain tissue with lower-nonlinearity cerebrospinal fluid. An increase in the parametric array signal magnitude relative to the baseline parametric array signal magnitude can indicate ventricular contraction, as successful shunt drainage replaces lower-nonlinearity cerebrospinal fluid with higher-nonlinearity brain tissue.

The portable device can include a user interface configured to display data indicative of changes in ventricular size based on the parametric array signal. The user interface can display real-time data on ventricular size and intracranial pressure changes. In some cases, the user interface can include a graphical user interface. The user interface can provide visual indicators of changes in ventricular size relative to a baseline state. The user interface can display alerts or notifications when the parametric array signal magnitude deviates from the baseline parametric array signal magnitude by a threshold amount. The user interface can provide feedback on the functionality of a hydrocephalus shunt by displaying data indicative of changes in ventricular volume corresponding to shunt drainage efficacy.

The portable device can be configured for pediatric use, leveraging natural acoustic windows such as fontanelles for improved imaging in infants. The portable device can be compatible with existing ultrasound imaging protocols, reducing additional training or workflow disruptions. The portable device can provide continuous or frequent monitoring capabilities for hydrocephalus patients in both clinical and outpatient settings. The portable device can facilitate routine, noninvasive follow-up, reducing the reliance on invasive intracranial pressure catheters.

The disclosed technology can be further understood according to the following clauses:
Clause 1: A system for monitoring intracranial conditions in a subject, comprising: an ultrasonic transducer configured to transmit ultrasound waves at a first frequency and a second frequency through a skull of the subject toward brain tissue and cerebrospinal fluid within the skull; a receiver configured to detect a parametric array signal generated by nonlinear interaction of the ultrasound waves at the first frequency and the second frequency within the brain tissue and the cerebrospinal fluid, wherein the parametric array signal comprises a difference frequency component corresponding to a difference between the first frequency and the second frequency; and a processor configured to determine a change in a ratio of the cerebrospinal fluid to the brain tissue based on a magnitude of the parametric array signal.
Clause 2: The system of clause 1, wherein the processor is further configured to correlate the change in the ratio of the cerebrospinal fluid to the brain tissue with a change in ventricular size within the skull.
Clause 3: The system of clause 2, wherein the processor is further configured to infer a change in intracranial pressure based on the change in ventricular size.
Clause 4: The system of clause 1, wherein the ultrasonic transducer comprises a holographic lens configured to focus the ultrasound waves through the skull.
Clause 5: The system of clause 4, wherein the holographic lens comprises a phase-engineered lens configured to compensate for skull-induced aberrations.
Clause 6: The system of clause 1, wherein the ultrasonic transducer has a configurable F-number to adjust a focus of the ultrasound waves based on anatomy of the subject.
Clause 7: The system of clause 1, wherein the receiver is positioned on an opposite side of the skull from the ultrasonic transducer in a through-transmission arrangement.
Clause 8: The system of clause 1, wherein the first frequency and the second frequency are in a megahertz range, and wherein the difference frequency component is in a kilohertz range.
Clause 9: The system of clause 1, further comprising a signal processing unit configured to demodulate and filter the parametric array signal received by the receiver.
Clause 10: The system of clause 1, wherein the processor is further configured to: establish a baseline parametric array signal magnitude; and compare a subsequently measured parametric array signal magnitude to the baseline parametric array signal magnitude to detect a change in ventricular size.
Clause 11: The system of clause 10, wherein a decrease in the parametric array signal magnitude relative to the baseline parametric array signal magnitude indicates ventricular expansion.
Clause 12: The system of clause 10, wherein an increase in the parametric array signal magnitude relative to the baseline parametric array signal magnitude indicates ventricular contraction.
Clause 13: The system of clause 1, wherein the processor is further configured to assess functionality of a hydrocephalus shunt based on the magnitude of the parametric array signal.
Clause 14: A method for monitoring intracranial conditions in a subject, comprising: transmitting, via an ultrasonic transducer, ultrasound waves at a first frequency and a second frequency through a skull of the subject toward brain tissue and cerebrospinal fluid within the skull; detecting, via a receiver, a parametric array signal generated by nonlinear interaction of the ultrasound waves at the first frequency and the second frequency within the brain tissue and the cerebrospinal fluid, wherein the parametric array signal comprises a difference frequency component corresponding to a difference between the first frequency and the second frequency; and determining, via a processor, a change in a ratio of the cerebrospinal fluid to the brain tissue based on a magnitude of the parametric array signal.
Clause 15: The method of clause 14, further comprising: establishing a baseline parametric array signal magnitude; and comparing a subsequently measured parametric array signal magnitude to the baseline parametric array signal magnitude to infer a change in ventricular size.
Clause 16: The method of clause 15, further comprising inferring a change in intracranial pressure based on the change in ventricular size.
Clause 17: The method of clause 14, wherein transmitting the ultrasound waves comprises focusing the ultrasound waves through the skull via a holographic lens coupled to the ultrasonic transducer.
Clause 18: The method of clause 14, wherein detecting the parametric array signal comprises receiving the parametric array signal at a location on an opposite side of the skull from the ultrasonic transducer.
Clause 19: The method of clause 14, further comprising continuously monitoring the magnitude of the parametric array signal over a period of time to track changes in ventricular size.
Clause 20: A portable device for noninvasive monitoring of cerebrospinal fluid dynamics in a subject, comprising: an ultrasonic transducer configured to transmit ultrasound waves at a first frequency and a second frequency through a skull of the subject; a receiver configured to detect a parametric array signal at a difference frequency generated by nonlinear interaction of the ultrasound waves within brain tissue and cerebrospinal fluid of the subject; a signal processing unit configured to analyze the parametric array signal to determine changes in a ratio of the cerebrospinal fluid to the brain tissue; and a user interface configured to display data indicative of changes in ventricular size based on the parametric array signal.

A number of implementations have been described. Nevertheless, it will be understood that various modifications may be made without departing from the spirit and scope of the disclosure. Accordingly, other implementations are within the scope of the following claims.

1. A system for monitoring intracranial conditions in a subject, comprising:
an ultrasonic transducer configured to transmit ultrasound waves at a first frequency and a second frequency through a skull of the subject toward brain tissue and cerebrospinal fluid within the skull;
a receiver configured to detect a parametric array signal generated by nonlinear interaction of the ultrasound waves at the first frequency and the second frequency within the brain tissue and the cerebrospinal fluid, wherein the parametric array signal comprises a difference frequency component corresponding to a difference between the first frequency and the second frequency; and
a processor configured to determine a change in a ratio of the cerebrospinal fluid to the brain tissue based on a magnitude of the parametric array signal.

2. The system of claim 1, wherein the processor is further configured to correlate the change in the ratio of the cerebrospinal fluid to the brain tissue with a change in ventricular size within the skull.

3. The system of claim 2, wherein the processor is further configured to infer a change in intracranial pressure based on the change in ventricular size.

4. The system of claim 1, wherein the ultrasonic transducer comprises a holographic lens configured to focus the ultrasound waves through the skull.

5. The system of claim 4, wherein the holographic lens comprises a phase-engineered lens configured to compensate for skull-induced aberrations.

6. The system of claim 1, wherein the ultrasonic transducer has a configurable F-number to adjust a focus of the ultrasound waves based on anatomy of the subject.

7. The system of claim 1, wherein the receiver is positioned on an opposite side of the skull from the ultrasonic transducer in a through-transmission arrangement.

8. The system of claim 1, wherein the first frequency and the second frequency are in a megahertz range, and wherein the difference frequency component is in a kilohertz range.

9. The system of claim 1, further comprising a signal processing unit configured to demodulate and filter the parametric array signal received by the receiver.

10. The system of claim 1, wherein the processor is further configured to:
establish a baseline parametric array signal magnitude; and
compare a subsequently measured parametric array signal magnitude to the baseline parametric array signal magnitude to detect a change in ventricular size.

11. The system of claim 10, wherein a decrease in the subsequently measured parametric array signal magnitude relative to the baseline parametric array signal magnitude indicates ventricular expansion.

12. The system of claim 10, wherein an increase in the subsequently measured parametric array signal magnitude relative to the baseline parametric array signal magnitude indicates ventricular contraction.

13. The system of claim 1, wherein the processor is further configured to assess functionality of a hydrocephalus shunt based on the magnitude of the parametric array signal.

14. A method for monitoring intracranial conditions in a subject, comprising:
transmitting, via an ultrasonic transducer, ultrasound waves at a first frequency and a second frequency through a skull of the subject toward brain tissue and cerebrospinal fluid within the skull;
detecting, via a receiver, a parametric array signal generated by nonlinear interaction of the ultrasound waves at the first frequency and the second frequency within the brain tissue and the cerebrospinal fluid, wherein the parametric array signal comprises a difference frequency component corresponding to a difference between the first frequency and the second frequency; and
determining, via a processor, a change in a ratio of the cerebrospinal fluid to the brain tissue based on a magnitude of the parametric array signal.

15. The method of claim 14, further comprising:
establishing a baseline parametric array signal magnitude; and
comparing a subsequently measured parametric array signal magnitude to the baseline parametric array signal magnitude to infer a change in ventricular size.

16. The method of claim 15, further comprising inferring a change in intracranial pressure based on the change in ventricular size.

17. The method of claim 14, wherein transmitting the ultrasound waves comprises focusing the ultrasound waves through the skull via a holographic lens coupled to the ultrasonic transducer.

18. The method of claim 14, wherein detecting the parametric array signal comprises receiving the parametric array signal at a location on an opposite side of the skull from the ultrasonic transducer.

19. The method of claim 14, further comprising continuously monitoring the magnitude of the parametric array signal over a period of time to track changes in ventricular size.

20. A portable device for noninvasive monitoring of cerebrospinal fluid dynamics in a subject, comprising:
an ultrasonic transducer configured to transmit ultrasound waves at a first frequency and a second frequency through a skull of the subject;
a receiver configured to detect a parametric array signal at a difference frequency generated by nonlinear interaction of the ultrasound waves within brain tissue and cerebrospinal fluid of the subject;
a signal processing unit configured to analyze the parametric array signal to determine changes in a ratio of the cerebrospinal fluid to the brain tissue; and
a user interface configured to display data indicative of changes in ventricular size based on the parametric array signal.
